(19) United States
(12) Patent Application Publication
(10) Pub. No.: US 2004/0077902 A1
(43) Pub. Date: Apr. 22, 2004

(54) Dicreatine maleate and method for making same

(76) Inventor: William Charles Llewellyn, Sound Beach, NY (US)

Correspondence Address: William Llewellyn, PO Box 1162, Sound Beach, NY, 11789, US

(21) Appl. No.: 10/274,203

(22) Filed: Oct. 18, 2002

(51) Int. Cl. A61K 31/205; C07C 279/10

(52) U.S. Cl. 562/580; 514/554

(57) ABSTRACT

This invention discloses the hydrosoluble creatine salt dicreatine maleate, and a method for manufacturing same. Creatine is a popular ergogenic aid, and is found most often in the form of creatine monohydrate. Creatine monohydrate is poorly soluble in water however, prompting this inventor to search for a more suitable form of creatine for nutritional supplementation. Dicreatine maleate offers a level of water solubility more than 12 fold better than creatine monohydrate.

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO A MICROFICHE APPENDIX

Not Applicable

### BACKGROUND OF THE INVENTION

Creatine is a naturally occurring metabolite found in the red muscle tissue of various animal species, including humans. Creatine plays an important role in the energy storage and expenditure of skeletal muscle, specifically via its metabolic precursor relationship to creatine phosphate. Creatine phosphate is used by the body to replenish levels of ATP (adenosine triphosphate), which is stored in muscle cells and used to provide energy through its conversion into ADP (adenosine diphosphate) during physical activity. Creatine phosphate is used to restore ADP to its original tri-phosphate energy-producing form. Muscle endurance is largely dependent on levels of available ATP.

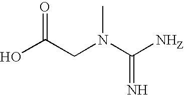

Creatine is found most commonly in nutritional supplements in the form of creatine monohydrate. This form of creatine offers some stability against the conversion of creatine to creatinine, an inactive byproduct, creating a more bioactive form of creatine. Being widely available, it is also the form of creatine most studied and shown to be an effective ergogenic aid in clinical trials with athletes. It is, however, not readily soluble in water, which causes some difficulties in producing a marketable and highly efficacious product. As of late many methods have been explored to manufacture more hydrosoluble salts of creatine, including citric and maleic salts.

U.S. Pat. No. 5,973,199 discloses a form of a creatine salt that is a combination of one mole of creatine monohydrate with one mole of maleic acid, i.e., a monocreatine maleate. Particularly, Example 2 of this patent discloses the combination of 0.10 moles of maleic acid with 0.10 moles of creatine monohydrate. A two-to-one ratio of creatine to maleic acid would be necessary for the production of a dicreatine salt. Such salt is not disclosed in patent 5,973,199.

It would be desirable to provide another form of creatine that is stable and has high water solubility. It would further be desirable to provide a form of creatine salt that provides more creatine per gram of weight than the monocreatine maleate salt.

### BRIEF SUMMARY OF THE INVENTION

The invention is a dicreatine maleate comprising two creatine cations per maleate dianion. The dicreatine maleate has a creatine content of approximately 69% by weight.

### BRIEF DESCRIPTION OF THE SEVERAL VIEWS OF THE DRAWING

Not Applicable

### DETAILED DESCRIPTION OF THE INVENTION

While this invention is susceptible of embodiment in many different forms, there is shown in the drawings and will herein be described in detail preferred embodiments of the invention with the understanding that the present disclosure is to be considered as an exemplification of the principles of the invention and is not intended to limit the broad aspect of the invention to the embodiments illustrated.

To obtain high water soluble organic salt of creatine, this patent deals exclusively with the formation of dicreatine maleate. By means of this procedure, the water solubility of creatine is increased by over 12 fold.

### EXAMPLE 1

The dicreatine maleate of the invention may be made as follows.

Add one gram-moles of maleic acid in 1,500 ml or organic alkane alcohol. Stir for approximately one hour cold until it is completely dissolved. Add two gram-moles of creatine monohydrate into the solution and allow it to react overnight. It is then filtered and washed with the same alkane alcohol. The alcohol is recovered for reuse.

The addition of an organic acid, e.g. acetic acid, into the solution can improve the reagent of the reaction and increase the yield.

The finished product is accordingly a dicreatine citrate, having two creatine cations per maleate dianion. An assay for the creatine content in the product gives a value of approximately 69%. The monocreatine maleate would give a value of approximately 53%.

I claim:

1. A dicreatine maleate, said dicreatine maleate comprising two creatine cations per maleate dianion.

2. The dicreatine maleate of claim 1, wherein said discreatine maleate has a creatine content of approximately 69% (wt.).
